(19) United States
(12) Patent Application Publication
(10) Pub. No.: US 2025/0019605 A1
(43) Pub. Date: Jan. 16, 2025

(54) AUTOTHERMAL CRACKING OF HYDROCARBONS

(71) Applicant: SHELL USA, INC., Houston, TX (US)

(72) Inventors: Ronald Jan SCHOONEBEEK, Amsterdam (NL); Dominik Johannes Michael UNRUH, Amsterdam (NL); Patrick Ivor Maurice VAN DER GULIK, Amsterdam (NL); Alouisius Nicolaas Renée BOS, Amsterdam (NL); Robert SCHOUWENAAR, Amsterdam (NL); Shauvik DE, Bangalore North (IN)

(21) Appl. No.: 18/711,890

(22) PCT Filed: Nov. 22, 2022
(86) PCT No.: PCT/EP2022/082705

§ 371 Date: May 21, 2024

(30) Foreign Application Priority Data
Dec. 28, 2021 (IN) 202141061196
Feb. 8, 2022 (EP) 22155545.1

(51) Int. Cl. C10G 69/06

(52) U.S. Cl. CPC C10G 69/06; C10G 2300/4006; C10G 2300/4018

(57) ABSTRACT

The invention relates to a process for producing olefins from a feed stream containing hydrocarbons by pyrolytic cracking of the hydrocarbons in an autothermal reactor. An oxygen containing stream and a hydrogen and/or methane containing stream are pre-heated outside the autothermal reactor. The pre-heated oxygen containing stream and the pre-heated hydrogen and/or methane containing stream are fed into a burner of the autothermal reactor. Steam is generated in a combustion zone of the autothermal reactor. A feed stream containing hydrocarbons is pre-heated outside the autothermal reactor and then fed into the autothermal reactor. The pre-heated feed stream is mixed with the steam generated in the combustion zone in a mixing and cracking zone of the autothermal reactor. The steam and the pre-heated feed stream containing hydrocarbons are fed into the mixing and cracking zone from substantially opposite directions. The hydrocarbons are pyrolytically cracked the to provide an effluent containing olefins.

### FIELD OF THE INVENTION

The present invention relates to a process for producing olefins from a feed stream containing hydrocarbons, by pyrolytic cracking of the hydrocarbons in an autothermal reactor.

### BACKGROUND OF THE INVENTION

Pyrolytic cracking of hydrocarbons in a cracker furnace is a petrochemical process that is widely used to produce olefins (such as ethylene, propylene, butylenes and butadiene) and optionally aromatics (such as benzene, toluene and xylene). Where such pyrolytic cracking is performed in the presence of dilution steam, this is referred to as “steam cracking”. The feed stream to such a pyrolytic cracking process may include one or more of ethane, propane, butane, liquefied petroleum gas (LPG), naphtha and hydrowax. In such a pyrolytic cracking process, the hydrocarbons containing stream is converted under the influence of heat, and substantially in the absence of oxygen, into an olefins containing effluent.

Although pyrolytic steam cracking in cracker furnaces is the industry standard for producing olefins, it has several disadvantages. The process produces large amounts of carbon dioxide. The heat that is needed to effect the pyrolytic cracking of the hydrocarbon stream is provided by the combustion of fuel gas, typically comprising hydrogen and methane, in a burner of the cracker furnace, i.e. the heat for the pyrolytic cracking is provided externally. Combustion of a fuel gas comprising hydrogen and methane results in the production of a flue gas comprising water and carbon dioxide. Generally, carbon dioxide from such flue gas may have to be emitted into the Earth's atmosphere and/or may have to be captured in another form thereby preventing such emission. A distinction can be made between Carbon Capture and Storage (CCS) and Carbon Capture and Use (CCU) which both involve carbon dioxide capture which is cumbersome, requiring additional equipment, and therefore relatively expensive. In addition, CCS further increases the general costs of chemicals manufacturing because of the required energy expenditure for compression and distribution to carbon dioxide storage.

In addition, the flue gas also comprises nitrogen oxide (NOx), which is a further undesirable by-product of the steam cracking reaction. NOX can be removed from the flue gas, for example by using a DeNOx system, but this necessitates an additional step in the process and increases the overall cost of the olefins manufacture.

Furthermore, notable amounts of coke are formed on the inside of the tubes/coils (carrying the hydrocarbon feed and where the pyrolytic cracking reaction takes place) suspended in the furnace, due to the provision of heat from outside of the furnace, which necessitates regular furnace shutdowns to remove the coke build-up from the tubes/coils. The tubes/coils are typically decoked using a mixture of steam and air. To help reduce the formation of coke, sometimes sulphur is added to the hydrocarbon feed stream. However, this then means that further treatment is required to remove H₂S from the off-gas process stream.

Moreover, aside from yielding the desired olefin products, depending on the hydrocarbon feed stream and reactor conditions used, the process can also yield (aside from coke) substantial amounts of other less desirable products such as methane, higher hydrocarbons, and heavy aromatics.

Furthermore, the conventional method of steam cracking hydrocarbons in a cracker furnace is extremely energy intensive and is associated with high operating expenses and high capital expenditure. The process requires a huge consumption of fuel and maintenance of large, expensive and complex cracking furnaces to supply the heat. For example, the tubes/coils used in the furnaces are very expensive and have a limited lifetime.

Therefore, it is an objective of the present invention to provide a process for the pyrolytic cracking of hydrocarbons that substantially reduces or avoids the production of carbon dioxide.

It is also an objective of the present invention to provide a process for the pyrolytic cracking of hydrocarbons that substantially reduces or avoids the formation of NOx.

It is a further objective of the present invention to provide a process for the pyrolytic cracking of hydrocarbons that substantially reduces or avoids the formation of coke and thus requires less reactor maintenance.

Additionally, it is an objective of the invention to provide increased selectivities and yields for the desired olefin product(s) compared to the selectivities and yields achieved by the conventional steam cracking process.

Further, it is an objective of the present invention to provide a process for the pyrolytic cracking of hydrocarbons to olefins, which process is efficient and affordable, and in particular has relatively low operating expenses, relatively low capital expenditure and relatively low energy demand.

These and other objectives will become apparent from the disclosure provided herein.

### SUMMARY OF THE INVENTION

The present invention relates to a process for producing olefins from a feed stream containing hydrocarbons by pyrolytic cracking of the hydrocarbons in an autothermal reactor, said process comprising:
pre-heating an oxygen containing stream and a hydrogen and/or methane containing stream outside the autothermal reactor;
feeding the pre-heated oxygen containing stream and the pre-heated hydrogen and/or methane containing stream into a burner of the autothermal reactor;
generating steam in a combustion zone of the autothermal reactor;
pre-heating a feed stream containing hydrocarbons outside the autothermal reactor;
feeding the pre-heated feed stream containing hydrocarbons into the autothermal reactor;
mixing the steam generated in the combustion zone with the pre-heated feed stream containing hydrocarbons in a mixing and cracking zone of the autothermal reactor, by feeding the steam and the pre-heated feed stream containing hydrocarbons into the mixing and cracking zone from substantially opposite directions, and pyrolytically cracking the hydrocarbons to provide an effluent containing olefins.

The step of feeding the pre-heated oxygen containing stream and the pre-heated hydrogen and/or methane containing stream into the burner of the autothermal reactor may further comprise feeding a pre-heated temperature moderator into the burner of the autothermal reactor.

The pre-heated temperature moderator may comprise steam and/or carbon dioxide.

The oxygen containing stream may be pre-heated to a temperature in the range of from about 200° C. to about 300° C.

The hydrogen and/or methane containing stream may be pre-heated to a temperature in the range of from about 350° C. to about 650° C.

The temperature moderator may be pre-heated to a temperature in the range of from about 350° C. to about 650° C.

The temperature of the steam generated in the combustion zone may be in the range of from about 1400° C. to about 1900° C., suitably about 1400° C. to about 1800° C.

The steam generated in the combustion zone may flow into the mixing and cracking zone at a velocity in the range of from about 100 m/s to about 400 m/s.

The feed stream containing hydrocarbons may flow into the mixing and cracking zone at a velocity in the range of from about 50 m/s to about 300 m/s.

The feed stream containing hydrocarbons may be pre-heated outside the autothermal reactor to a temperature in the range of from about 200° C. to about 650° C. Alternatively, the feed stream containing hydrocarbons pre-heated outside the reactor may be further heated inside the reactor to a temperature in the range of from about 200° C. to about 650° C. through indirect heat exchange.

The feed stream containing hydrocarbons may be further heated inside the reactor through indirect heat exchange between the effluent containing olefins and the feed stream containing hydrocarbons in an effluent zone of the reactor.

The feed stream containing hydrocarbons may comprise any one or more of ethane, propane, butane, liquefied petroleum gas (LPG), naphtha, hydrowax, gas oil, bio-naphtha and bio-diesel. Methane may be added to the afore-mentioned feed stream containing hydrocarbons.

The effluent containing olefins may undergo further downstream processing and/or separation in a steam cracker unit.

The process of the present invention is advantageous in that a reduced amount of carbon dioxide or no or very little carbon dioxide is produced during the process. This is due to the heat for the cracking reaction being produced inside the reactor using feed streams that generate no or little carbon dioxide, for example when hydrogen and oxygen are fed to the burner of the reactor. Where oxygen and methane are fed to the burner, or oxygen and hydrogen and methane are fed to the burner, then some carbon dioxide may be produced, but the amount of carbon dioxide produced will still be lower than that produced using conventional pyrolytic cracking of hydrocarbons in a cracker furnace, which uses the external combustion of hydrogen and methane as the heat source.

The process of the present invention is also beneficial in that it avoids or substantially reduces the production of NOx. Where methane is fed to the burner (in combination with oxygen or in combination with oxygen and hydrogen), then some NOx may be produced, but the amount of NOx produced will still be lower than that produced using conventional pyrolytic cracking of hydrocarbons in a cracker furnace. The reduction or elimination of NOx production helps to reduce the cost of the olefins manufacture and minimize the release of this pollutant into the atmosphere.

A significant further benefit of the process of the invention is that no or little coke is formed during cracking. Since the formation of coke is substantially or altogether avoided, the reactor can run for much longer periods of time without interruption, and if any coke is formed, it can be easily and quickly removed from the reactor. In addition, since no or little coke is formed during the process of the present invention, there is no need to add sulphur to the hydrocarbon feed stream and thus there is also no need for any treatment to remove H₂S during the process.

The rapid and efficient mixing of the steam and the feed stream comprising hydrocarbons in the mixing and cracking zone of the reactor enables high cracking temperatures and fast cracking times that in turn leads to increased selectivities and high yields for the desired olefins, particularly when compared to those obtained using conventional cracking of hydrocarbons in a cracker furnace.

Overall, the process of the present invention involves lower capital expenditure compared to conventional steam cracking processes using cracker furnaces. This is due to various factors including the simpler design of the autothermal reactor and much shorter reaction times. The process is also advantageous in that lower operating expenses are incurred due in part to the high selectivities achieved and higher operating temperatures used.

### BRIEF DESCRIPTION OF THE DRAWINGS

FIG. 1 shows a schematic representation of an autothermal reactor for use in an embodiment of the process of the present invention.

FIG. 2 shows a schematic representation of an alternative configuration of an autothermal reactor for use in an embodiment of the process of the present invention.

FIG. 3 shows a schematic representation of another alternative configuration of an autothermal reactor for use in an embodiment of the process of the present invention.

FIG. 4 shows the oven and reactor configuration used in the high temperature and low residence time ethane steam cracking experiment of Example 1.

FIG. 5 shows the temperature profile of the 2.39 mm Al₂O₃ reactor with a solid 2 mm O.D. Al₂O₃ insert and a nitrogen flow of 10 Nl/hr, placed inside the 5 cm microheater, in the high temperature and low residence time ethane steam cracking experiment of Example 1.

FIG. 6 shows a Computational Fluid Dynamics (CFD) simulation of a comparative mixing configuration where ethane is introduced into the reactor via slits on the side of the reactor as discussed in Example 2.

FIG. 7 shows a Computational Fluid Dynamics (CFD) simulation of a mixing configuration according to an embodiment of the process of the present invention where ethane is introduced into the mixing and cracking zone of the reactor via a lance as discussed in Example 2.

### DETAILED DESCRIPTION OF THE INVENTION

The process of the present invention comprises multiple steps. In addition, said process may comprise one or more intermediate steps between consecutive steps. Further, said process may comprise one or more additional steps preceding the first step and/or following the last step. For example, in a case where said process comprises steps a), b) and c), said process may comprise one or more intermediate steps between steps a) and b) and between steps b) and c). Further, said process may comprise one or more additional steps preceding step a) and/or following step c).

While the process of the present invention and the streams used in the process are described in terms of “comprising”, “containing” or “including” one or more various described steps and components, respectively, they can also “consist essentially of” or “consist of” said one or more various described steps and components, respectively.

In the context of the present invention, in a case where a stream comprises two or more components, these components are to be selected in an overall amount not to exceed 100%.

Further, where upper and lower limits are quoted for a property then a range of values defined by a combination of any of the upper limits with any of the lower limits is also implied.

In general terms the present invention provides a method for the cracking of hydrocarbons to olefins in an autothermal reactor.

For the purposes of promoting an understanding of the principles of the invention, reference will now be made to the embodiments illustrated in the accompanying drawings, which are described in more detail below. The embodiments disclosed herein are not intended to be exhaustive or limit the invention to the precise form disclosed in the following detailed description. The invention includes any alterations and further modifications in the illustrated devices and described methods and further applications of the principles of the invention as set forth in the claims.

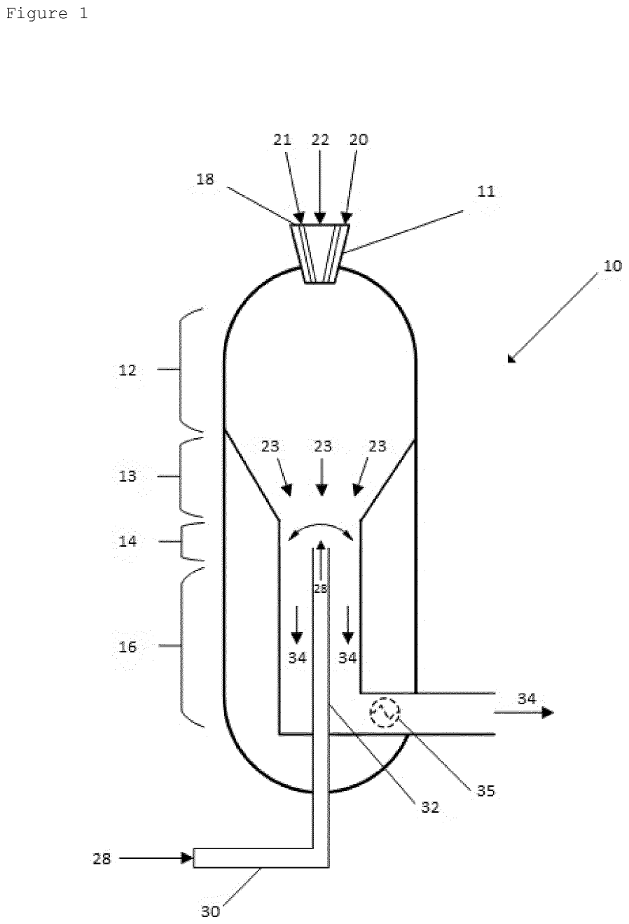

FIG. 1 shows a representation of an autothermal reactor 10 for use in a process for producing olefins from a feed stream containing hydrocarbons by pyrolytic cracking of the hydrocarbons according to an embodiment of the present invention.

The autothermal reactor 10 comprises a burner 11, a combustion zone 12, a contraction zone 13, a mixing and cracking zone 14, and an effluent zone 16.

The burner 11 has an inlet section 18 through which an oxygen containing stream 20 and a hydrogen and/or methane containing stream 22 are fed into the burner 11. The inlet section 18 may comprise multiple inlets, one for each of the respective streams to be fed into the burner 11.

In the present invention, the hydrogen and/or methane containing stream preferably contains hydrogen. Further, said hydrogen containing stream may also contain methane. More preferably, said hydrogen containing stream consists of hydrogen. The amount of methane in the hydrogen and/or methane containing stream may be of from 0 to 15 vol. % or may be at most 10 vol. % or at most 5 vol. % or at most 1 vol. % or at most 0.5 vol. %, based on the total amount of hydrogen and methane. Further, said amount of methane in the hydrogen and/or methane containing stream may be at least 0.5 vol. % or at least 1.5 vol. % or at least 3 vol. %.

Hydrogen used in the hydrogen and/or methane containing stream 22, whether used alone or in combination with methane, can be any suitable source of hydrogen, including conventional hydrogen (so-called “grey” hydrogen), hydrogen sustainably produced through renewable power electrolysis (so-called “green” hydrogen), hydrogen produced from hydrocarbons in a process like steam methane reforming in combination with carbon capture and storage (so-called “blue” hydrogen), or otherwise produced hydrogen.

Methane used in the hydrogen and/or methane containing stream 22, whether used alone or in combination with hydrogen, can be any suitable source of methane, including conventional methane as well as methane from renewable sources (so-called “green” methane).

The oxygen containing stream 20 and the hydrogen and/or methane containing stream 22 are each pre-heated prior to being fed into the burner 11. Any known means for pre-heating the streams can be used. The oxygen containing stream 20 is typically pre-heated to a temperature in the range of from about 200° C. to about 300° C. Suitably, the oxygen containing stream 20 can be pre-heated to a temperature of at least 200° C., suitably at least 220° C., suitably at least 240° C. Suitably, the oxygen containing stream 20 can be pre-heated to a temperature of at most 300° C., suitably at most 280° C., suitably at most 260° C. The hydrogen and/or methane containing stream 22 is typically pre-heated to a temperature in the range of from about 350° C. to about 650° C. Suitably, the hydrogen and/or methane containing stream 22 can be pre-heated to a temperature of at least 350° C., suitably at least 400° C., suitably at least 450° C. Suitably, the hydrogen and/or methane containing stream 22 can be pre-heated to a temperature of at most 650° C., suitably at most 600° C., suitably at most 550° C. The temperature to which these two streams are pre-heated can be varied accordingly depending on the desired temperature of the steam 23 generated in the combustion zone 12.

Other components can be fed into the burner 11 in addition to the oxygen containing stream 20 and the hydrogen and/or methane containing stream 22. For example, a temperature moderator 21, such as steam and/or carbon dioxide, can be fed into the burner 11 to help regulate the temperature of the steam 23 that results from the combustion of oxygen and hydrogen, or oxygen and methane, or all three of oxygen, hydrogen and methane (when a mixture of hydrogen and methane is used in stream 22) in the combustion zone 12. The temperature of this additional temperature moderator 21, e.g. steam and/or carbon dioxide, can be varied accordingly depending on the desired temperature of the steam 23 generated in the combustion zone 12. Typically, the temperature moderator 21 (whether it be steam, carbon dioxide or a mixture of both, or indeed any other suitable temperature moderator) is pre-heated to a temperature in the range of from about 350° C. to about 650° C. before being fed into the burner 11. Suitably, the temperature moderator 21 can be pre-heated to a temperature of at least 350° C., suitably at least 400° C., suitably at least 450° C. Suitably, the temperature moderator 21 can be pre-heated to a temperature of at most 650° C., suitably at most 600° C., suitably at most 550° C. The temperature moderator 21 can be fed to the burner 11 alone or in combination with the oxygen containing stream 20 and/or the hydrogen and/or methane containing stream 22. In the embodiment shown in FIG. 1, purely for simplicity purposes, the optional temperature moderator 21 is shown as being fed into the burner 11 separately to the oxygen containing stream 20 and the hydrogen and/or methane containing stream 22.

The addition of steam and/or carbon dioxide, or other inert gas, to the burner 11 can also help to prevent the oxygen and the hydrogen and/or methane from reacting in close vicinity of the burner inlet section 18.

The oxygen and hydrogen, or the oxygen and methane, or the oxygen and hydrogen and methane (depending on the composition of the feed stream 22) combust in the combustion zone 12 leading to a high flame temperature and the formation of steam 23, substantially free of oxygen. Within the present specification, steam “substantially free of oxygen” means that the steam contains of from 0 to 10,000 parts per million of volume (ppmv) of oxygen or may contain oxygen in an amount of at most 5,000 ppmv or at most 1,000 ppmv or at most 500 ppmv or at most 10 ppmv. In the present invention, such steam substantially free of oxygen may be provided by feeding hydrogen in an amount which is higher than the stoichiometric molar amount needed for the reaction of hydrogen with oxygen, for example 1-5% or 1-3% higher. The temperature of the steam 23 generated in the combustion zone 12 can vary depending on the type of hydrocarbon feedstock to be cracked. Typically, the temperature of the steam 23 generated in the combustion zone 12 is in the range of from about 1400° C. to about 1900° C., suitably about 1400° C. to about 1800° C., depending on the type of hydrocarbon feedstock to be cracked. Suitably, the temperature of the steam 23 generated in the combustion zone 12 can be at least 1400° C., suitably at least 1450° C., suitably at most 1900° C., suitably at most 1800° C., suitably at most 1750° C. For example, where ethane is used as the hydrocarbon feedstock, the temperature of the steam 23 generated in the combustion zone 12 can suitably be in the range of from about 1650° C. to about 1750° C.

This high-temperature steam 23, generated within the autothermal reactor 10, is the heat source that is used to effect the pyrolytic cracking of hydrocarbons in the process of the present invention. The generation of heat in this manner is advantageous over conventional pyrolytic cracking of hydrocarbons in a cracker furnace, using the external combustion of hydrogen and methane as the heat source, because no or very little carbon dioxide is produced when oxygen and hydrogen are fed to the burner 11 (without the presence of methane or other hydrocarbon being co-fed). In case oxygen and methane are fed to the burner 11, or oxygen and hydrogen and methane are fed to the burner 11, then some carbon dioxide may be produced, but the amount of carbon dioxide produced will still be lower than that produced using conventional pyrolytic cracking of hydrocarbons in a cracker furnace.

The high-temperature steam 23 generated in the combustion zone 12 flows into the contraction zone 13, which as shown in FIG. 1 is located below the combustion zone 12. The contraction zone 13 is narrower in width than the combustion zone 12 and narrows downwards along its length to ensure that the high-temperature steam 23 flows at a very high velocity downwards towards the mixing and cracking zone 14 of the reactor 10.

A wide variety of hydrocarbon feedstocks can be used in the process of the present invention. The feed stream containing hydrocarbons 28 contains saturated hydrocarbons and may optionally contain unsaturated hydrocarbons. Further, before the feed stream containing hydrocarbons 28 is subjected to the process of the present invention, it may be gaseous or may be in liquid form. Suitably, the feed stream 28 may contain C2+ hydrocarbons. Suitably, the feed stream containing hydrocarbons 28 may include any one or more of ethane, propane, butane, liquefied petroleum gas (LPG), naphtha, hydrowax and gas oil. Bio-derived and synthetic hydrocarbons can also be used, such as bio-naphtha and bio-diesel.

It is possible for some methane to be added to the hydrocarbon feed stream 28. In this instance, the methane can be considered as a secondary hydrocarbon feedstock, with the above-mentioned hydrocarbons (e.g. C2+ hydrocarbons; or the ethane, propane, butane, liquefied petroleum gas (LPG), naphtha, hydrowax, gas oil, bio-naphtha and bio-diesel) being the primary hydrocarbon feedstock. The methane can be added simultaneously with the primary hydrocarbon feedstock (e.g. it can be mixed in with the primary hydrocarbon feedstock), or it can be introduced into the reactor prior to introduction of the primary hydrocarbon feedstock. Typically, the optional additional methane constitutes a relatively small proportion of the total hydrocarbon feed stream 28.

The feed stream containing hydrocarbons 28 is pre-heated prior to being fed into the reactor 10. Any known means for pre-heating the feed stream can be used. The temperature to which the feed stream comprising hydrocarbons 28 is pre-heated depends partly on the type of hydrocarbon feedstock being used. It also depends on whether or not any further heating of the feed stream containing hydrocarbons 28 takes place inside the reactor 10 prior to contacting of the feed stream containing hydrocarbons 28 with the high-temperature steam 23 (also referred to herein as the “steam stream”) in the mixing and cracking zone 14 (which is discussed in further detail below). Ultimately, what is important is the temperature of the feed stream containing hydrocarbons 28 immediately before or as it contacts the steam stream 23 in the mixing and cracking zone 14. Typically, the temperature of the feed stream containing hydrocarbons 28 just before it contacts the steam 23 in the mixing and cracking zone 14 is in the range of from about 200° C. to about 650° C., depending on the type of hydrocarbon feedstock being used. Suitably, the temperature of the feed stream containing hydrocarbons 28 just before it contacts the steam 23 in the mixing and cracking zone 14 can be at least 200° C., suitably at least 250° C., suitably at most 650° C., suitably at most 600° C. For example, where ethane is used as the hydrocarbon feedstock, the temperature of the feed stream containing ethane can suitably be in the range of from about 550° C. to about 650° C. This may mean that the feed stream containing hydrocarbons 28 is pre-heated to the desired temperature (i.e. the temperature immediately before or just as it contacts the steam 23 in the mixing and cracking zone 14), e.g. to a temperature in any of the afore-mentioned ranges, depending on the hydrocarbon feedstock being used, prior to being fed into the reactor 10, if no significant further heating of the feed stream containing hydrocarbons 28 takes place inside the reactor 10 prior to contacting of the feed stream containing hydrocarbons 28 with the steam stream 23 in the mixing and cracking zone 14. If, as discussed in further detail below and as per the embodiment of the invention shown in FIG. 1, for example, further heating of the feed stream containing hydrocarbons 28 does take place inside the reactor 10 prior to contacting of the feed stream containing hydrocarbons 28 with the steam stream 23 in the mixing and cracking zone 14, then the feed stream containing hydrocarbons 28 can be pre-heated to a lower temperature than the desired temperature (i.e. the temperature immediately before or just as it contacts the steam 23 in the mixing and cracking zone 14) prior to being fed into the reactor 10, since it will be heated up further in the reactor 10 prior to mixing with the steam stream 23. The extent to which the feed stream containing hydrocarbons 28 is further heated inside in the reactor will depend on various factors, including the mechanism by which the feed stream containing hydrocarbons 28 is further heated (e.g. by indirect heat exchange), the length of time for which it is exposed to additional heat and the type of hydrocarbon feedstock being used. For example, the additional heating inside the reactor may typically increase the temperature of the pre-heated feed stream containing hydrocarbons by between about 10° C. and about 200° C. So, the feed stream containing hydrocarbons can typically be pre-heated outside the reactor to a temperature between about 10° C. and about 200° C. lower than the desired temperature of about 200° C. to about 650° C. when the feed stream containing hydrocarbons enters the mixing and cracking zone 14. In either scenario (i.e. whether there is any significant further heating of the hydrocarbon feedstock 28 in the reactor 10 or not), the temperature of the pre-heated feed stream containing hydrocarbons 28 is lower than the temperature of the steam 23 generated in the combustion zone 12, both when it is fed into the reactor 10 and immediately before or as it contacts the steam 23 in the mixing and cracking zone 14.

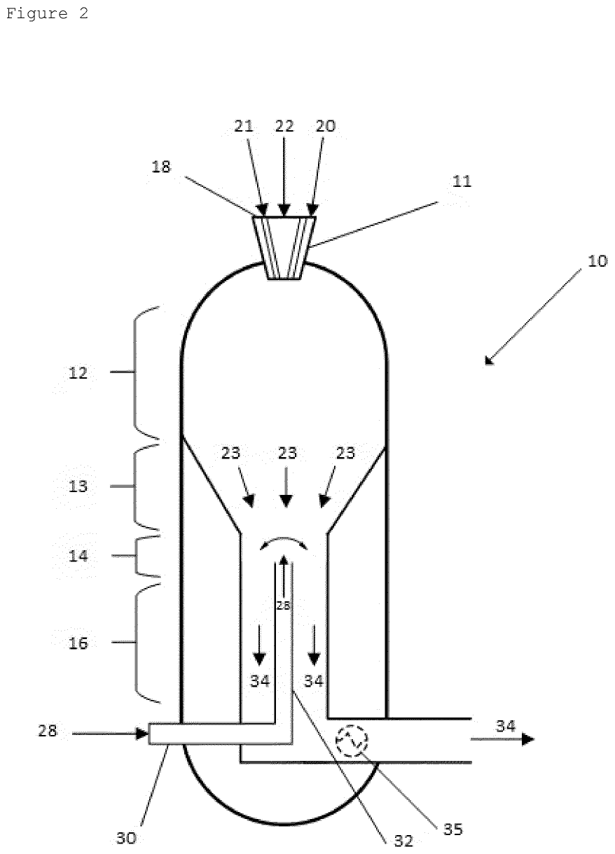

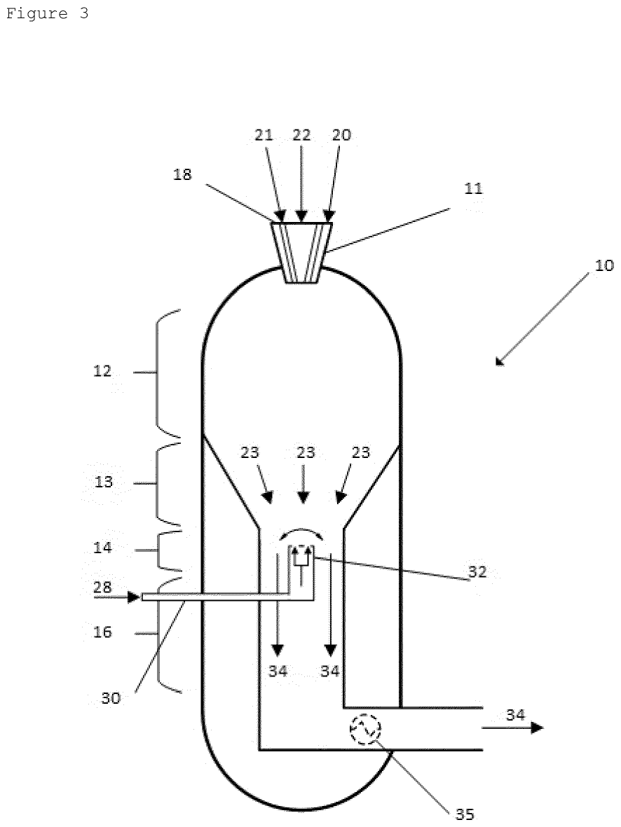

The feed stream containing hydrocarbons 28 can be fed into the reactor 10 through inlet 30. In the reactor configuration shown in FIG. 1, inlet 30, for simplicity reasons, is shown as a side-arm, whereas in practice alternative configurations of inlet and other means of introducing the hydrocarbon containing feed stream 28 into the reactor 10 are possible and included within the scope of the present invention. Also, in practice there may be more than one inlet or side-arm present to introduce the hydrocarbons 28 into the reactor 10. In FIG. 1, the inlet 30 is depicted as being located beneath and to the side of the reactor 10. FIGS. 2 and 3 show alternative configurations of reactor 10, where the inlet 30 is shown as a single side-arm protruding from a side wall of the reactor 10. The location of the inlet can be any suitable location that allows the hydrocarbon containing feed stream 28 to be introduced into the reactor in a suitable manner so as to realise the advantages of the present invention. In the reactor configuration shown in FIG. 1, the feed stream containing hydrocarbons 28, once introduced through inlet 30, typically by high speed injection, flows upwards towards the mixing and cracking zone 14 through a narrow inner tube or lance 32. The narrow width of the lance 32 ensures the feed stream containing hydrocarbons 28 flows at high velocity, typically at a velocity in the range of from about 50 m/s to 300 m/s, towards the mixing and cracking zone 14 of the reactor 10. In the reactor configuration shown in FIG. 1, the lance 32 extends from beneath the reactor, upwards through the effluent zone 16, and terminates at the mixing and cracking zone 14.

In the mixing and cracking zone 14, the steam stream 23 from the contraction zone 13 is contacted with the feed stream containing hydrocarbons 28 from the lance 32 and the two streams mix. Both streams are flowing at high velocity and thus mixing occurs rapidly, although preferably the steam stream 23 is moving at a higher velocity than the feed stream containing hydrocarbons 28. Typically, the steam stream 23 is flowing at a velocity in the range of from about 100 m/s to about 400 m/s, suitably in the range from about 150 to about 300 m/s. Suitably, the steam stream 23 is flowing at a velocity in the range of from about 50 m/s to about 150 m/s higher than that of the feed stream containing hydrocarbons 28.

In the reactor configuration shown in FIG. 1, the use of the lance 32 ensures that the feed stream containing hydrocarbons 28 flows upwards towards the mixing and cracking zone 14, such that the steam stream 23 and the feed stream containing hydrocarbons 28 are flowing towards the mixing and cracking zone 14 in substantially opposite directions, i.e. they are flowing counter-currently, and that they collide and contact each other substantially head-on in the mixing and cracking zone 14. This substantially opposite or counter-current high-velocity flow of the two streams, i.e. the steam stream 23 and the feed stream containing hydrocarbons 28, has been found to lead to extremely fast and efficient mixing of the two streams. For example, the opposite flow of the two high-velocity streams has been found to lead to much faster and more efficient mixing of the two streams compared to when the feed stream containing hydrocarbons 28 enters the mixing and cracking zone 14 and is contacted with the steam stream 23 perpendicular to the flow of the steam stream 23, e.g. in a configuration where the inlet for the entry of the feed stream containing hydrocarbons into the reactor leads directly into the mixing and cracking zone without the use of an upwardly extending narrow tube or lance (see Example 2).

The steam stream 23 and the feed stream containing hydrocarbons 28 can be directly opposite streams moving towards one another and fed into the mixing and cracking zone 14 to contact and mix with one another, or they can be substantially opposite streams, i.e. the streams can be slightly off-set and do not have to be fed into the mixing and cracking zone 14 from precisely opposite directions. Thus, within the present specification, “substantially opposite directions” for the steam and the pre-heated feed stream containing hydrocarbons when feeding into the mixing and cracking zone, covers both (i) directly or precisely opposite directions, that is to say 100% opposite directions (directions with a difference of 180°), and (ii) directions which deviate from said 100% opposite directions to some extent. In the present invention, the deviation from said 100% opposite directions may be of from 0 to 20° or may be at most 15° or at most 10° or at most 5° or at most 3° or at most 1°.

The rapid mixing due to the opposing or counter-current flow of the two high-velocity streams has the benefit of avoiding back-mixing of the feed stream containing hydrocarbons 28 in the steam 23 in the mixing and cracking zone 14. Such back-mixing can mean that the hydrocarbon cracks for too long at high temperatures, causing a build-up of coke and other undesired reactions.

Optionally, steam can be added to the feed stream containing hydrocarbons 28 to help avoid coke formation in the lance 32 and/or to help increase the hydrocarbon to olefin conversion after mixing.

Optionally, there can be a device present in the reactor 10 that causes the steam 23 to swirl in the contraction zone 13 to assist with rapid and efficient mixing of the two opposing streams. Also, optionally, there can be a nozzle or multiple outlets (see FIG. 3) present at the tip of the lance (at the mixing and cracking zone 14) to change the local injection velocity of the hydrocarbon feed stream 28 as it enters the mixing and cracking zone 14.

Mixing of the high-temperature steam stream 23 (which is typically at a temperature in the range of from about 1400° C. to about 1900° C., suitably about 1400° C. to about 1800° C., when it reaches the mixing and cracking zone 14) with the cooler feed stream containing hydrocarbons 28 causes the feed steam containing hydrocarbons 28 to heat up. Thus, the high-temperature steam 23 is the heat source that is used to effect the pyrolytic cracking of the hydrocarbons in the process of the present invention.

The cracking temperatures, resulting from the rapid mixing of the high-temperature steam 23 and the cooler feed stream containing hydrocarbons 28, in the process of the present invention for producing olefins from a feed stream containing hydrocarbons are much higher than the cracking temperatures used in conventional steam cracking in a cracker furnace. Typically, the cracking temperatures are up to a few hundred degrees higher (for example, about 200° C. to about 400° C. higher) than conventional steam cracking in a cracker furnace, which typically takes place at around 800-850° C. Thus, in the present invention, the cracking temperature in the mixing and cracking zone may be of from 1,000 to 1,250° C. In the present invention, the pyrolytic cracking in the mixing and cracking zone of the autothermal reactor is preferably carried out without using a catalyst.

The temperature of the steam 23 output from the combustion zone 12 and the temperature of the feed stream containing hydrocarbons 28 when it reaches the mixing and cracking zone 14 are optimised and selected so as to achieve the desired cracking temperatures for the chosen hydrocarbon feedstock.

The cracking times in the process of the present invention for producing olefins from a feed stream containing hydrocarbons are much shorter than the cracking times typically observed in conventional steam cracking in a cracker furnace. Typically, the cracking times using the process of the present invention are in the range of from about 1 millisecond (ms) to about 20 milliseconds (ms), depending on the hydrocarbon feedstock to be cracked, so about two orders of magnitude lower than the cracking times typically observed in conventional steam cracking in a cracker furnace.

The high cracking temperatures and the short cracking times achieved using the process of the present invention surprisingly provide better yields and selectivities for the desired olefins compared to conventional steam cracking of hydrocarbons in a cracker furnace. Furthermore, no or very little coke is produced at such high cracking temperatures and short cracking times.

The mixing of the feed stream containing hydrocarbons 28 and the steam stream 23 in the mixing and cracking zone 14 is so rapid and thorough that mixing and cracking largely occur simultaneously. As such, a majority of the hydrocarbons crack whilst in the mixing and cracking zone 14, although some cracking may also take place in the effluent zone 16 (so-called “after-cracking”).

In the reactor configuration shown in FIG. 1, the effluent 34 containing olefins, i.e. the effluent stream containing the cracked products, flows downwards through the effluent zone 16 of the reactor 10 around the outside of the lance 32.

This exemplified counter-current flow arrangement not only provides for the feed stream containing hydrocarbons 28 and the steam stream 23 to collide and meet as substantially opposing streams and mix head-on in the mixing and cracking zone 14, but it also allows for indirect heat-exchange to take place between the cooler feed stream containing hydrocarbons 28 flowing upwards through the lance 32 and the resultant effluent 34 flowing downwards around the outside of the lance 32 in the effluent zone 16. The resultant effluent 34 flowing downwards around the outside of the lance 32 is at a higher temperature than the feed stream containing hydrocarbons 28 inside the lance 32 and cools as it flows downwards towards the base of the reactor 10. Any remaining hydrocarbons being cracked in the effluent zone 16 will also be at a higher temperature than the feed stream containing hydrocarbons 28 inside the lance 32. The temperature at which the cracked effluent 34 containing the desired olefin products leaves the effluent zone 16 depends somewhat on the hydrocarbon feedstock used. Typically, the effluent 34 leaves the effluent zone 16 at a temperature of around 800° C. As discussed earlier, when such a hydrocarbon injection arrangement as shown in FIG. 1 is present in the reactor 10, the feed stream containing hydrocarbons 28 advantageously only needs to be pre-heated outside the reactor 10 to a temperature that is lower than the desired target temperature of the feed stream containing hydrocarbons 28 as it reaches the mixing and cracking zone 14, because heat transfer will take place along the length of the lance 32 from the warmer effluent 34 containing the desired olefin products flowing downwards outside the lance 32 to the cooler feed stream containing hydrocarbons 28 flowing upwards inside the lance 32. Lower pre-heating temperatures for the feed stock containing hydrocarbons 28 reduces the risk of undesired cracking in a pre-heater and also reduces the cost of heating the feed stream containing hydrocarbons 28 outside the reactor 10 prior to introducing it into the reactor 10. The feed stream containing hydrocarbons 28 flowing upwards through the lance 32 also helps to rapidly cool the effluent 34 in the effluent zone 16.

A longer lance 32 will provide for increased indirect heat transfer between the cooler feed stock containing hydrocarbons 28 flowing upwards in the lance 32 and the effluent 34 flowing downwards around the lance 32. Although in practice there may be a limit on the maximum desirable length of the lance depending on engineering and construction considerations, such as vibration of the lance. Supports can be used to stabilise the lance to reduce/prevent vibration of the lance.

The reactor configuration shown in FIG. 2 has a somewhat shorter lance than that shown in FIG. 1, and the reactor configuration shown in FIG. 3 has a much shorter lance than that shown in FIG. 1. In the configuration shown in FIG. 3, there will be much less heat transfer than in the configuration shown in FIG. 1 as the lance does not extend the full length of the effluent zone 16.

As discussed earlier, the indirect heat-exchange flow arrangement shown in FIGS. 1 to 3 is not essential to the process of the present invention, but it is a beneficial feature that arises due to the feeding/injection of hydrocarbons 28 through the lance 32 to create opposing streams (i.e. the steam stream 23 and the feed stream containing hydrocarbons 28) in the mixing and cracking zone 14.

Optionally, there may be a separate heat exchanger 35 present in the reactor. Typically, such a separate heat exchanger would be present in the effluent zone 16 of the reactor 10. The heat exchanger 35 may be used to pre-heat the feed stream containing hydrocarbons 28 outside the autothermal reactor. Such a heat exchanger also may be used to pre-heat the oxygen containing stream 20 and/or the hydrogen and/or methane containing stream 22 before they enter the burner 11.

As mentioned earlier, the process of the present invention provides improved olefin yields and selectivities compared to those obtainable with conventional steam cracking of hydrocarbons in a cracker furnace. As a consequence, fewer less desirable secondary products are made.

From the effluent zone 16, an effluent 34 is obtained that comprises olefins which may include one or more of ethylene, propylene, butylenes and butadiene, and hydrogen, water and carbon dioxide, and that may comprise aromatics (as produced in the cracking process) which may include one or more of benzene, toluene and xylene. The specific products obtained depend on the composition of the hydrocarbon feed stream, the hydrocarbon-to-steam ratio, the cracking temperature and the cracking time.

Where acetylene is produced as a secondary product, it can be hydrogenated to ethylene in a further catalytic step.

Depending on the composition of the resultant effluent 34 and the desired products, at least a portion of the effluent 34 output from the autothermal reactor 10 can undergo further downstream processing and/or separation in a conventional steam cracker unit.

The process of the present invention can be operated at higher pressures than those used in conventional steam cracking of hydrocarbons in a cracker furnace due to the higher cracking temperatures used in the present process. Higher operating pressures reduce the capital expenditure associated with the autothermal reactor.

The ratio of steam to hydrocarbons used in the process of the present invention is higher than that used in the conventional steam cracking of hydrocarbons in a cracker furnace. This contributes to the improved olefin yields and selectivities observed when using the process of the present invention and also the reduction or elimination of coke formation observed when using the process of the present invention.

The invention is further illustrated by the following Examples.

### EXAMPLES

### Example 1—Cracking of Ethane to Ethylene

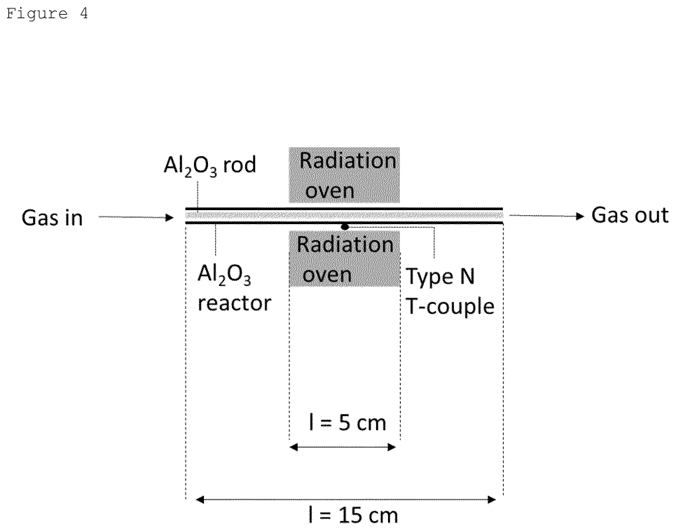

In the experiments of Example 1, a gas stream having the composition as described in Table 1 was fed to a horizontally oriented, tubular, alumina reactor placed in a radiation oven. FIG. 4 depicts the oven and reactor configuration described in more detail below.

The alumina reactor tube had an inner diameter of 2.39 mm and a length of 150 mm. To achieve the desired low residence time of the gas in the hot reaction zone, a solid alumina rod was placed inside the reactor. This solid alumina rod had an outer diameter of 2 mm and a length of 15 cm. A section of 5 cm of the reactor tube was placed in a temperature controlled radiation oven. The radiation oven had a length of 5 cm; the isothermal zone of the oven was only 1.5 cm. The isothermal zone subsequently had a free volume of 0.02 ml.

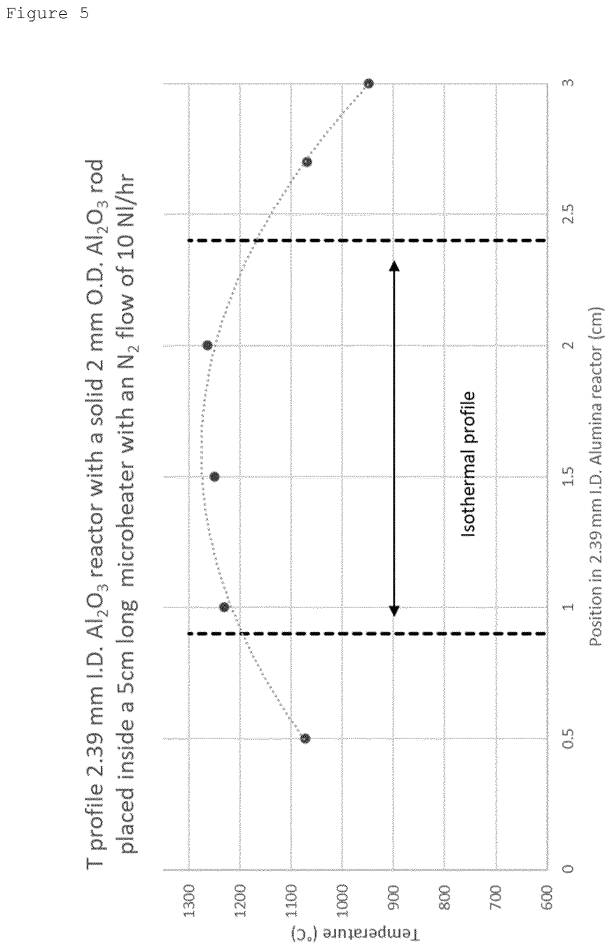

The temperature of this isothermal zone was measured with a type N thermocouple having a 0.5 mm outer diameter and a length of 300 mm. This thermocouple was mounted on the outside of the alumina reactor. A temperature profile along the length of the alumina reactor was measured, this was done at several temperature settings. FIG. 5 shows an example of a typical temperature profile at an oven setpoint of 1250° C.

Table 1 summarises the conditions of 5 experiments as well as two reference conditions pertaining to conventional cracker operation. Experiments were done at several temperatures and different inlet gas flows (residence times) and compositions. Flow rates are reported in Nl/hr where “Nl” stands for “normal litre” as measured at standard temperature and pressure.

The reported residence times were calculated on the basis of the flow rate at actual average temperature and pressure in the isothermal zone and the free reactor volume of that isothermal zone.

TABLE 1

| | | | | | | H₂O | N₂ | C₂H₆ |
|---|---|---|---|---|---|---|---|---|
| | Temp | Residence | H₂O | N₂ | C₂H₆ | % | % | % |
| Exp | (° C.) | time msec | Nl/hr | Nl/hr | Nl/hr | Vol | Vol | Vol |
| 1 | 1050 | 2.3 | 2.91 | 0.52 | 5.5 | 32.6 | 5.8 | 61.6 |
| 2 | 1150 | 2.1 | 2.91 | 0.52 | 5.5 | 32.6 | 5.8 | 61.6 |
| 3 | 1250 | 2 | 2.91 | 0.52 | 5.5 | 32.6 | 5.8 | 61.6 |
| 4 | 1150 | 1.9 | 1.49 | 0.52 | 8.25 | 14.5 | 5.1 | 80.4 |
| 5 | 1250 | 1.8 | 1.49 | 0.52 | 8.25 | 14.5 | 5.1 | 80.4 |
| Ref1⁽*⁾ | 850 | 400 | 1.49 | 0.52 | 8.25 | 14.5 | 5.1 | 80.4 |
| Ref2⁽*⁾ | 860 | 200 | 1.49 | 0.52 | 8.25 | 14.5 | 5.1 | 80.4 |

⁽*⁾These are reference conditions reflecting conventional cracker conditions

The experimental results for the conditions of Table 1 are given in Table 2. The total off gas flow rate was calculated, using nitrogen as internal standard, from the results of on-line gas chromatograph (GC) analyzers for the feed and product gas streams. From this total off gas flow the individual component flows were calculated in Nl/hr. For each experiment the selectivity and ethane conversion was determined by the formulae given below, where:
the flowrate of each component is denoted by the component formula;
the subscript “_feed” denotes the flow rate of the component in the combined feed stream; all the other flow rates pertain to the product stream;
C3 selectivity is the sum of the components propane, propylene, propyne and propadiene; C4 selectivity is the sum of the components butane, butenes and butadienes; C7⁺ is the sum of mainly toluene, C8 aromatics and C9 aromatics;
all the values are in Nl/hr and the calculated conversion xᵢ and selectivity sᵢ are % on a molar basis:

| | |
|---|---|
| C₂H₆ conversion | xC₂H₆ = (1-C₂H₆/C₂H₆_feed) *100 |
| CH₄ selectivity | sC₂H₄ = (CH₄/(C₂H₆_feed − C₂H₆)) *100 |
| CO selectivity | sCO = (CH₄/(C₂H₆_feed − C₂H₆)) *100 |
| C₂H₄ selectivity | sC₂H₄ = (C₂H₄/(C₂H₆_feed − C₂H₆)) *100 |
| C₂H₂ selectivity | sC₂H2 = (C₂H₂/(C₂H₆_feed − C₂H₆)) *100 |
| C3 selectivity | sC3 = (C3/(C₂H₆_feed − C₂H₆)) *100 |
| C4 selectivity | sC3 = (C3/(C₂H₆_feed − C₂H₆)) *100 |
| Benzene | sC₆H₆ = (C₆H₆/(C₂H₆_feed − C₂H₆)) *100 |
| selectivity | |
| C7⁺ selectivity | sC7⁺ = (C7⁺ aromatics/(C₂H6_feed − C₂H₆)) *100 |

TABLE 2

| | xC₂H₆ | sCH₄ | sCO | sC₂H₄ | sC₂H₂ | sC3 | sC4 | sC₆H₆ | |
|---|---|---|---|---|---|---|---|---|---|
| Exp. | (%) | (%) | (%) ⁽¹⁾ | (%) | (%) | (%) | (%) | (%) | sC7⁺ |
| 1 | 13.2 | 2.49 | 0.00 | 95.3 | 0.43 | 0.29 | 1.45 | 0.00 | 0.00 |
| 2 | 41.8 | 4.26 | 0.01 | 92.2 | 1.39 | 1.10 | 0.75 | 0.22 | 0.05 |
| 3 | 74.6 | 6.89 | 0.02 | 85.2 | 4.85 | 1.49 | 0.17 | 1.10 | 0.29 |
| 4 | 20.5 | 3.30 | 0.00 | 94.1 | 0.40 | 0.72 | 1.50 | 0.00 | 0.00 |
| 5 | 54.8 | 5.60 | 0.01 | 89.8 | 2.04 | 1.44 | 0.57 | 0.49 | 0.11 |
| Ref1⁽*⁾ | 60 | 4.00 | | 88.4 | 0.93 | 1.61 | 3.17 | 0.81 | 0.92 |
| Ref2⁽*⁾ | 70 | 5.00 | | 85.4 | 1.29 | 1.76 | 3.57 | 1.40 | 1.02 |

⁽*⁾These are calculated using the cracker model “Spyro” which is commonly used to calculate the cracker performance at a certain condition
⁽¹⁾ In none of the experiments, carbon dioxide (CO₂) was detected in the effluent.

As it is well known that the selectivity of the cracking process is dependent on the ethane conversion, performances should best be compared at equal or similar conversion levels.

Because acetylene can be routinely selectively hydrogenated to ethylene, the sum of the selectivity of ethylene plus acetylene is also an important performance indicator. The experimental results in Table 2 above demonstrate that this sum of ethylene and acetylene selectivity is always significantly higher in the short contact time and high temperature experiments (Experiments 1-5), compared to the cracking in a conventional steam cracker (Ref1 and Ref2).

Also, Table 2 demonstrates that the selectivity towards the less desired higher hydrocarbons (from C3 to C7⁺ aromatics) in Experiments 1-5 is significantly lower compared to that of Ref1 and Ref2.

Moreover, in the short contact time cracking experiments (Exps 1-5) no carbon or coke formation was observed in the reactor tube, whereas coke formation is known to occur significantly for Ref1 and Ref2 conditions.

### Example 2—Assessment of Mixing Configurations

Computational Fluid Dynamics (CFD) simulations of various potential commercial scale mixing configurations were performed focusing on achieving the required fast mixing of the hot steam and colder hydrocarbon, i.e. the mixing time scale being at least as short as the time of the cracking reaction. The hydrocarbon used in the simulations was ethane.

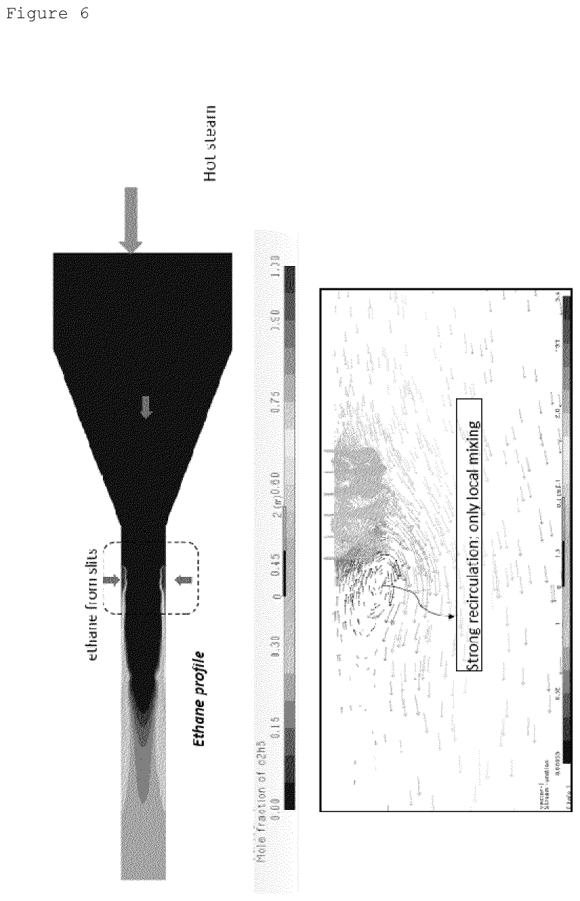

FIG. 6 shows the results for an autothermal reactor where the ethane is introduced into the reactor via 6 slits on the side of the reactor. This would be an obvious choice for those skilled in the art after applying design rules from e.g. jet theory.

FIG. 6 shows that despite having applied these design rules and injecting the ethane at high velocity sideways into the hot steam, the mixing is very non-uniform and local circulation patterns are observed. If applied in practice, from the results in Example 1 it follows that in these non-uniform mixing zones there will be residence times (much) longer than the short residence times aimed for and hence reduction of the selectivity for ethylene (and acetylene) and likely significant carbon formation as seen in conventional crackers operating at these longer residence times.

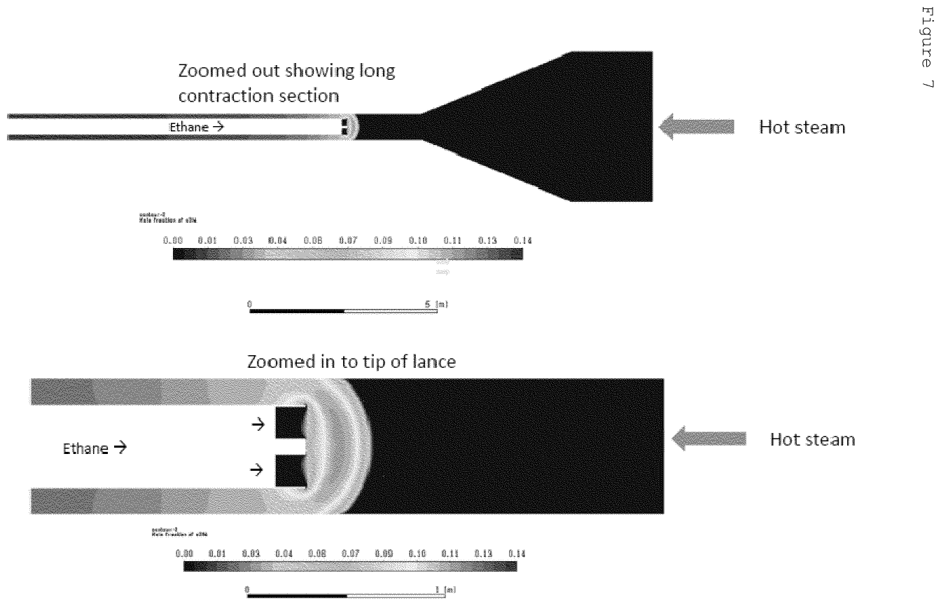

FIG. 7 shows the results of a mixing configuration according to an embodiment of the invention, i.e. introducing the ethane and steam in a counter-current/opposing stream fashion using a lance for introduction of the ethane at high velocity into the mixing and cracking zone of the autothermal reactor. As shown, the mixing is fast and uniform and occurs in a very small region near the outlet of the lance where the two streams (the hot steam and the colder ethane) collide head-on. No circulation patterns are observed. FIG. 7 also shows that a major part of the reaction takes place in that small region near the outlet of the lance with the desired high temperature near the tip of the lance and that in the effluent (after-cracking) zone the temperature is already low enough to prevent the loss in selectivity if exposed at longer residence times and high temperature such as in FIG. 6 for the side injection configuration.

In both said simulations (FIGS. 6 and 7), the temperature and velocity of the steam stream when flowing into the mixing and cracking zone were 1834° C. and 105 m/s, respectively. Further, the temperature and velocity of the ethane stream when flowing into the mixing and cracking zone were 600° C. and 90 m/s, respectively. The cracking temperature in the mixing and cracking zone was 1,100° C.

1. A process for producing olefins from a feed stream containing hydrocarbons by pyrolytic cracking of the hydrocarbons in an autothermal reactor, said process comprising:
pre-heating an oxygen containing stream and a hydrogen and/or methane containing stream outside the autothermal reactor;
feeding the pre-heated oxygen containing stream and the pre-heated hydrogen and/or methane containing stream into a burner of the autothermal reactor;
generating steam in a combustion zone of the autothermal reactor;
pre-heating a feed stream containing hydrocarbons outside the autothermal reactor;
feeding the pre-heated feed stream containing hydrocarbons into the autothermal reactor;
mixing the steam generated in the combustion zone with the pre-heated feed stream containing hydrocarbons in a mixing and cracking zone of the autothermal reactor, by feeding the steam and the pre-heated feed stream containing hydrocarbons into the mixing and cracking zone from substantially opposite directions, and pyrolytically cracking the hydrocarbons to provide an effluent containing olefins.

2. The process according to claim 1, wherein the step of feeding the pre-heated oxygen containing stream and the pre-heated hydrogen and/or methane containing stream into the burner of the autothermal reactor further comprises feeding a pre-heated temperature moderator into the burner of the autothermal reactor.

3. The process according to claim 2, wherein the pre-heated temperature moderator comprises steam and/or carbon dioxide.

4. The process according to claim 1, wherein the oxygen containing stream is pre-heated to a temperature in the range of from about 200° C. to about 300° C.

5. The process according to claim 1, wherein the hydrogen and/or methane containing stream is pre-heated to a temperature in the range of from about 350° C. to about 650° C.

6. The process according to claim 1, wherein the temperature moderator is pre-heated to a temperature in the range of from about 350° C. to about 650° C.

7. The process according to claim 1, wherein the temperature of the steam generated in the combustion zone is in the range of from about 1400° C. to about 1900° C.

8. The process according to claim 1, wherein the steam generated in the combustion zone flows into the mixing and cracking zone at a velocity in the range of from about 100 m/s to about 400 m/s.

9. The process according to claim 1, wherein the feed stream containing hydrocarbons flows into the mixing and cracking zone at a velocity in the range of from about 50 m/s to about 300 m/s.

10. The process according to claim 1, wherein the feed stream containing hydrocarbons is pre-heated outside the autothermal reactor to a temperature in the range of from about 200° C. to about 650° C.

11. The process according to claim 1, wherein the feed stream containing hydrocarbons pre-heated outside the reactor is further heated inside the reactor to a temperature in the range of from about 200° C. to about 650° C. through indirect heat exchange.

12. The process according to claim 11, wherein the feed stream containing hydrocarbons is further heated inside the reactor through indirect heat exchange between the effluent containing olefins and the feed stream containing hydrocarbons in an effluent zone of the reactor.

13. The process according to claim 1, wherein the feed stream containing hydrocarbons comprises any one or more of ethane, propane, butane, liquefied petroleum gas (LPG), naphtha, hydrowax, gas oil, bio-naphtha and bio-diesel.

14. The process according to claim 13, further comprising adding methane to the feed stream containing hydrocarbons.

15. The process according to claim 1, wherein at least a portion of the effluent containing olefins undergoes further downstream processing and/or separation in a steam cracker unit.
